(19) United States
(12) Patent Application Publication
(10) Pub. No.: US 2025/0186809 A1
(43) Pub. Date: Jun. 12, 2025

(54) ULTRASOUND TREATMENT DEVICE AND METHOD FOR CONTROLLING SAME

(71) Applicant: LG ELECTRONICS INC., Seoul (KR)

(72) Inventors: Sangwon KIM, Seoul (KR); Yongju YANG, Seoul (KR); Gyuseog HONG, Seoul (KR); Suwon SEO, Seoul (KR); Munseong KANG, Seoul (KR); Seungyoon SHIN, Seoul (KR); Dongwon KIM, Seoul (KR)

(73) Assignee: LG ELECTRONICS INC., Seoul (KR)

(21) Appl. No.: 18/843,919

(22) PCT Filed: Nov. 30, 2022
(86) PCT No.: PCT/KR2022/019211

§ 371 Date: Sep. 4, 2024

(51) Int. Cl. A61N 7/02; A61N 7/00

(52) U.S. Cl. CPC A61N 7/02; A61N 2007/0008

(57) ABSTRACT

An ultrasound treatment device comprising: a grip part; a head part coupled to one side of the grip part; an ultrasound generation part located on one side of the head part; a sensor module that detects whether one surface of the head part is in contact with the skin; and a control unit that controls the operation of the ultrasound generation part depending on whether the sensor module has detected contact, wherein the sensor module includes: an LED; an illuminance sensor disposed adjacent to the LED; and a partition between the LED and the illuminance sensor, wherein the illuminance sensor detects contactlessly when at least one of reflected light from the LED or external light is detected. The ultrasound treatment device can accurately detect whether the ultrasound treatment device is in contact with a user's skin by using not only external light but also light emitted from the LED through the sensor module including the LED, the illuminance sensor, and the partition.

### TECHNICAL FIELD

The present disclosure relates to an ultrasonic treatment device and a method for controlling the same that allow ultrasound to be irradiated only when the ultrasonic treatment device and user's skin are in complete contact with each other during an ultrasound treatment process.

### BACKGROUND

Recently, interest in skin care and obesity treatment is increasing day by day, and various medical and beauty devices for the skin care and the obesity treatment are being developed. For example, various skin care devices are being developed for patients who wish to undergo a face lifting or skin tightening procedure.

A non-invasive ultrasound device that has recently been in the spotlight is an ultrasonic treatment device that uses high intensity focused ultrasound (HIFU). This type of ultrasonic treatment device includes an ultrasonic treatment device that irradiates the high intensity focused ultrasound to shallow skin tissues for the skin care treatment to perform the skin lifting or skin tightening procedure, and an ultrasonic treatment device that irradiates the high intensity focused ultrasound to a subcutaneous fat layer for the obesity treatment to burn or melt fatty tissues.

In this regard, when using the high intensity focused ultrasound, it is very important to ensure that the high intensity focused ultrasound is irradiated while the ultrasonic treatment device is in complete contact with a treatment area.

For example, for the obesity treatment, the high intensity focused ultrasound should be irradiated only to the subcutaneous fat layer, which is a target layer, in a skin layer composed of a stratum corneum, an epidermis, a dermis, the subcutaneous fat layer, and a muscle layer to burn or melt and decompose the fat tissues.

When the high intensity focused ultrasound is irradiated while the ultrasonic treatment device is not in proper contact with the treatment area, not only will the ultrasound not be properly irradiated to the subcutaneous fat layer, but the high intensity focused ultrasound will also be incorrectly focused on a skin layer other than the subcutaneous fat layer, which may cause serious side effects, such as burns on the user's skin.

Therefore, in the case of the procedure using the high intensity focused ultrasound, to prevent the above-mentioned problems, means is needed to sense whether the ultrasonic treatment device is in contact with the user's skin and allow the ultrasound to be irradiated only when the complete contact is made.

Generally, an illuminance sensor is a sensor that senses light. Further, whether the ultrasonic treatment device is in contact with the user's skin may be sensed using the illuminance sensor. For example, when there is a spacing between the ultrasonic treatment device and the user's skin, external light may enter through the spacing, and whether the contact is made may be determined by sensing external light via the illuminance sensor.

However, the illuminance sensor is not able to properly sense light in a dark environment. In this case, even though there is no actual contact between the ultrasonic treatment device and the user's skin, the illuminance sensor may not be able to sense light and thus may determine that the contact has been made. Then, the ultrasonic treatment device irradiates the ultrasound without being in contact with the user's skin, causing the above-mentioned problem. In other words, there are limitations in determining whether the contact with the skin is made using the illuminance sensor alone.

### SUMMARY

### Technical Problem

The present disclosure provides an ultrasonic treatment device and a method for controlling the same. More specifically, the present disclosure is to provide an ultrasonic treatment device and a method for controlling the same that may accurately sense whether user's skin is in contact using light emitted from an LED as well as external light via a sensor module composed of the LED, an illuminance sensor, and a partition wall.

In addition, the present disclosure is to provide an ultrasonic treatment device and a method for controlling the same that allow ultrasound to be irradiated only when one surface of a head is in complete contact with user's skin by sensing whether the user's skin is in contact via each of a plurality of sensor modules located along an outer edge of the one surface of the head and spaced apart from each other.

The problems to be solved in the present disclosure are not limited to the problems mentioned above, and other problems not mentioned may be clearly understood by those skilled in the art in the technical field to which the present disclosure belongs from the description below.

### Technical Solutions

Provided is an ultrasonic treatment device including a gripping portion, a head coupled to one side of the gripping portion, an ultrasonic generator located on one surface of the head, a sensor module that senses whether the one surface of the head is in contact with skin, and a controller that controls operation of the ultrasonic generator based on whether the one surface of the head is in contact with the skin sensed by the sensor module, wherein the sensor module includes an LED, an illuminance sensor disposed adjacent to the LED, and a partition wall located between the LED and the illuminance sensor, wherein the illuminance sensor senses that the one surface of the head is not in contact with the skin when at least one of reflected light of the LED and external light is sensed.

The sensor module may include a plurality of sensor modules located along an outer edge of the one surface of the head and spaced apart from each other.

The controller may stop the operation of the ultrasonic generator when at least one of the respective illuminance sensors of the plurality of sensor modules senses that the one surface of the head is not in contact with the skin.

The partition wall may be painted black so as not to affect the sensing, by the illuminance sensor, of whether the one surface of the head is in contact with the skin.

The sensor module may have the partition wall having one end located on the one surface of the head, and the LED and the illuminance sensor may be located with vertical level differences from the one end of the partition wall.

The sensor module may include a first plate-shaped member vertically coupled to the partition wall, wherein the first plate-shaped member transmits light emitted from the LED therethrough, and a second plate-shaped member vertically coupled to the partition wall, wherein the second plate-shaped member transmits reflected light of the LED or external light therethrough.

The ultrasonic generator may include a first vibrator configured to generate high intensity focused ultrasound.

The ultrasonic generator may further include a second vibrator in a planar type.

Provided is a method for controlling an ultrasonic treatment device including operating an LED and an illuminance sensor of each of a plurality of sensor modules, sensing whether one surface of a head is in contact with skin via the plurality of sensor modules, and controlling operation of an ultrasonic generator based on whether the one surface of the head is in contact with the skin sensed by each of the plurality of sensor modules, wherein the sensing of whether the one surface of the head is in contact with the skin includes sensing, by the illuminance sensor, that the one surface of the head is not in contact with the skin when at least one of reflected light of the LED and external light is sensed.

The controlling of the operation of the ultrasonic generator may include stopping the operation of the ultrasonic generator when at least one of the respective illuminance sensors of the plurality of sensor modules senses that the one surface of the head is not in contact with the skin.

The method may further include outputting a notification to a user via a screen or by voice when it is sensed that the one surface of the head is not in contact with the skin.

### Advantageous Effects

The ultrasonic treatment device and the method for controlling the same according to the present disclosure may accurately sense whether the user's skin is in contact using light emitted from the LED as well as external light via the sensor module composed of the LED, the illuminance sensor, and the partition wall.

In addition, the ultrasound may be irradiated only when the one surface of the head is in complete contact with the user's skin by sensing whether the user's skin is in contact via each of the plurality of sensor modules located along the outer edge of the one surface of the head and spaced apart from each other.

Additional scope of applicability of the present disclosure will become apparent from the detailed description below. However, various changes and modifications within the spirit and scope of the present disclosure may be clearly understood by those skilled in the art, so that the detailed description and specific embodiments, such as preferred embodiments, of the present disclosure should be understood as being given by way of example only.

### BRIEF DESCRIPTION OF THE DRAWINGS

FIG. 1 is a block diagram of an ultrasonic treatment device according to an embodiment of the present disclosure.

FIG. 2 is a diagram showing an overall shape of an ultrasonic treatment device according to an embodiment of the present disclosure.

FIG. 3 is a bottom view of a head of an ultrasonic treatment device according to an embodiment of the present disclosure.

FIG. 4 is a cross-sectional view of a sensor module of an ultrasonic treatment device according to an embodiment of the present disclosure.

FIGS. 5 and 6 are diagrams for illustrating a scheme of sensing whether there is a contact with skin via a sensor module of an ultrasonic treatment device according to an embodiment of the present disclosure.

FIG. 7 is a diagram for illustrating a method for controlling an ultrasonic treatment device according to an embodiment of the present disclosure.

FIG. 8 is a diagram for illustrating a method for controlling an ultrasonic treatment device in FIG. 7 in more detail.

### BEST MODE

Description will now be given in detail according to exemplary embodiments disclosed herein, with reference to the accompanying drawings. The same or equivalent components may be provided with the same reference numbers, and description thereof will not be repeated. As used herein, the suffixes “module” and “part” are added or used interchangeably to facilitate preparation of this specification and are not intended to suggest distinct meanings or functions. In describing embodiments disclosed in this specification, relevant well-known technologies may not be described in detail in order not to obscure the subject matter of the embodiments disclosed in this specification. In addition, it should be noted that the accompanying drawings are only for easy understanding of the embodiments disclosed in the present specification, and should not be construed as limiting the technical spirit disclosed in the present specification. As such, the present disclosure should be construed to extend to any alterations, equivalents and substitutes in addition to those which are particularly set out in the accompanying drawings.

Although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, it will be understood that when an element is referred to as being “directly connected with” another element, there are no intervening elements present.

A singular representation may include a plural representation unless it represents a definitely different meaning from the context.

The terms such as “include” or “have” used herein are intended to indicate that features, numbers, steps, operations, elements, components, or combinations thereof used in the following description exist and it should be thus understood that the possibility of existence or addition of one or more different features, numbers, steps, operations, elements, components, or combinations thereof is not excluded.

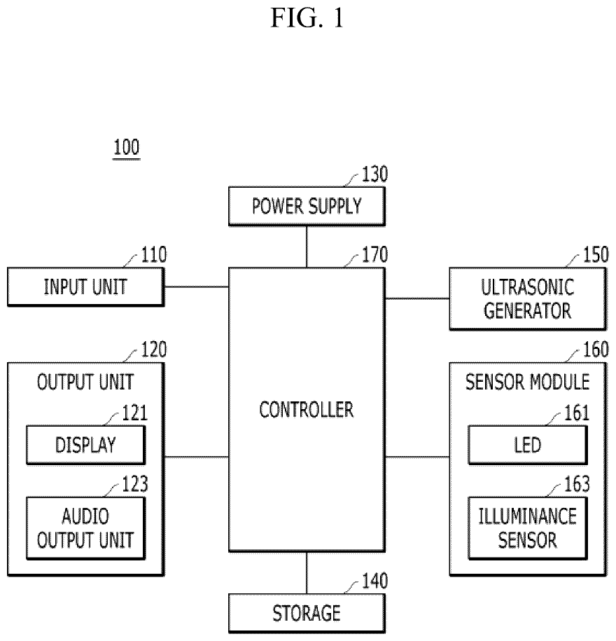

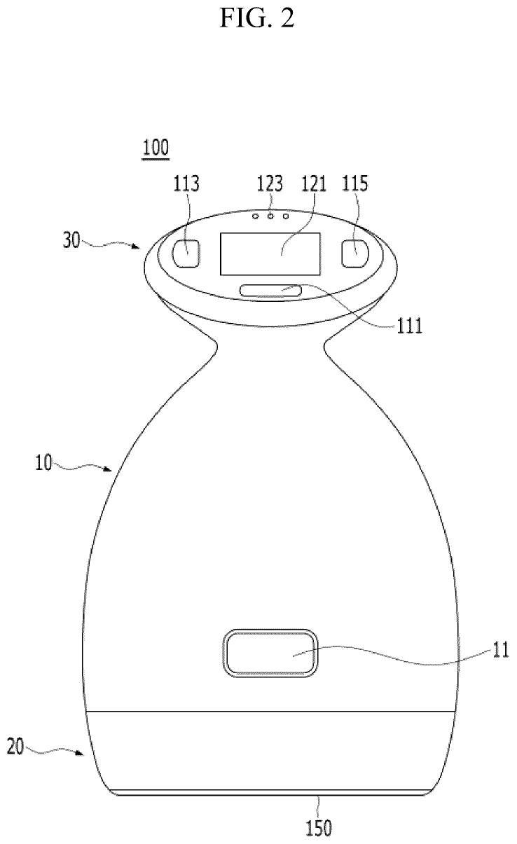

FIG. 1 is a block diagram of an ultrasonic treatment device 100 according to an embodiment of the present disclosure. FIG. 2 is a diagram showing an overall shape of the ultrasonic treatment device 100 according to an embodiment of the present disclosure.

The ultrasonic treatment device 100 according to an embodiment of the present disclosure may include an input unit 110, an output unit 120, a power supply 130, storage 140, an ultrasonic generator 150, a sensor module 160, and a controller 170.

In this regard, the input unit 110 may include a touch pad, a physical button, and the like, thereby receiving various user commands related to operation of the ultrasonic treatment device 100 and transmitting a control signal corresponding to the input command to the controller 170.

Referring to FIG. 2 together, in the ultrasonic treatment device 100 according to an embodiment of the present disclosure, the input unit 110 may include a power button 111, a mode selection button 113, and an operation button 115. Further, the input unit 110 may be located on a manipulator 30 disposed in a gripping portion 10. Recently, the number of ultrasonic treatment devices 100 in a form of minimizing the externally exposed physical buttons has increased. Accordingly, the power button 111, the mode selection button 113, and the operation button 115 described above may be omitted, and the externally exposed physical buttons may be minimized via remote control devices such as a remote controller.

In this regard, the power button 111 is a button that enables turning on or off the ultrasonic treatment device 100 itself of the present disclosure. Further, the user may set a treatment area, an operation time, timer setting, an ultrasonic intensity level, and the like via the mode selection button 113. In addition, the operation button 115 may transmit an operation signal of the ultrasonic generator 150 to the controller 170 such that ultrasound may be irradiated to the treatment area based on settings made by a user via mode selection.

The output unit 120, which is to generate an output related to vision, hearing, or tactile sensation, may include a display 121 and an audio output unit 123. Referring to FIG. 2 together, in the ultrasonic treatment device 100 according to an embodiment of the present disclosure, the output unit 120 may be located on the manipulator 30 disposed in the gripping portion 10.

In this regard, the display 121 may constitute a mutual layer structure with a touch sensor or may be integrally formed therewith. Additionally, the audio output unit 123 may include a receiver, a speaker, a buzzer, and the like.

The power supply 130 may supply power throughout the ultrasonic treatment device 100. Therefore, the power may be supplied to the controller 170, the display 121, the audio output unit 123, the ultrasonic generator 150, and the sensor module 160.

Specifically, the power supply 130 may include a converter (not shown) that converts AC power to DC power and a Dc/Dc converter (not shown) that converts a level of the DC power.

In one example, the power supply 130 receives the power from the outside and distributes the power to the respective components. The power supply 130 may use a scheme of supplying the AC power by being directly connected to an external power source, or may include a power supply 130 that includes a battery so as to be charged and used.

In the former case, the power supply 130 is used by being connected to a cable, so that the power supply 130 is difficult to move or has a limited range of movement. In the latter case, the power supply 130 is free to move, but a weight and a volume thereof increase as much as those of the battery, and the power supply 130 should be connected directly to a power cable or should be coupled to a charging stand (not shown) that supplies the power for a certain period of time for charging.

The charging stand may be connected to the ultrasonic treatment device 100 via a terminal exposed to the outside, or the built-in battery may be charged when it is close to the charging stand using a wireless scheme.

The storage 140 may store a program for processing and controlling each signal in the controller 170, and may store a signal-processed image, voice, or data signal. For example, the storage 140 may store application programs designed to perform various tasks that may be processed by the controller 170, and may selectively provide some of the stored application programs upon request from the controller 170.

The program or the like stored in the storage 140 is not particularly limited as long as it may be executed by the controller 170. The storage 140 may also perform a function for temporary storage of the image, the video, or the data signal.

FIG. 1 shows an embodiment in which the storage 140 is formed separately from the controller 170, but the scope of the present disclosure may not be limited thereto, and the storage 140 may be included within the controller 170.

The storage 140 may include at least one of a volatile memory (e.g., a DRAM, a SRAM, a SDRAM, and the like), a non-volatile memory (e.g., a flash memory, a hard disk drive (HDD), a solid-state drive (SSD), and the like).

The ultrasonic generator 150 may output the ultrasound such that the ultrasound may be irradiated to the treatment area. In particular, the ultrasonic generator 150 may include outputting high intensity focused ultrasound.

Referring to FIG. 2 together, in the ultrasonic treatment device 100 according to an embodiment of the present disclosure, the ultrasonic generator 150 may be located on one surface of a head 20. Therefore, the ultrasonic generator 150 may output the ultrasound in a direction the head 20 is facing or in a direction similar thereto. For example, when one surface of the head 20 is facing user's abdomen, the ultrasonic generator 150 outputs the ultrasound toward the user's abdomen.

In this regard, the ultrasonic generator 150 may output the ultrasound with an intensity based on settings made by the user via the mode selection button 113 described above based on the operation time.

The sensor module 160 includes an LED 161 and an illuminance sensor 163. In this regard, the illuminance sensor 163 may sense light. Further, whether there is a contact between the ultrasonic treatment device 100 and user's skin, which is the treatment area, may be sensed via the sensor module 160. A scheme of sensing whether the contact is made will be described in detail below.

The controller 170 may control all or some of the components included in the ultrasonic treatment device 100. For example, the controller 170 may control the input unit 110, the output unit 120, the ultrasonic generator 150, and the sensor module 160.

In particular, in the ultrasonic treatment device 100 according to an embodiment of the present disclosure, the controller 170 may control the operation of the ultrasonic generator 150 based on whether the device is in contact with the user's skin sensed by the sensor module 160. Accordingly, the operation of the ultrasonic generator 150 may be controlled such that the ultrasonic generator 150 outputs the ultrasound only when the ultrasonic treatment device 100 and the user's skin are in complete contact with each other.

The block diagram of the ultrasonic treatment device 100 shown in FIG. 1 is only a block diagram for one embodiment of the present disclosure. Respective components of the block diagram may be integrated, added, or omitted based on a specification of the ultrasonic treatment device 100 that is actually implemented.

That is, as needed, two or more components may be combined into one component, or one component may be subdivided into two or more components. In addition, a function performed by each block is to illustrate the embodiment of the present disclosure, and a specific operation or device does not limit the scope of rights of the present disclosure.

FIG. 2 is a diagram showing an overall shape of the ultrasonic treatment device 100 according to an embodiment of the present disclosure, including the gripping portion 10 held by the user, the head 20 coupled to one side of the gripping portion 10, and the ultrasonic generator 150 located on the one surface of the head 20.

In this regard, the gripping portion 10 may include a detachment button 11 formed on a side surface thereof to couple or separate the head 20. That is, the head 20 may be detachably coupled to the gripping portion 10. In this regard, the controller 170 may identify whether the head 20 is coupled or separated by a preset identification code.

Additionally, as described above, the gripping portion 10 may have the manipulator 30. Additionally, the manipulator 30 may include the power button 111, the mode selection button 113, the operation button 115, the display 121, and the audio output unit 123.

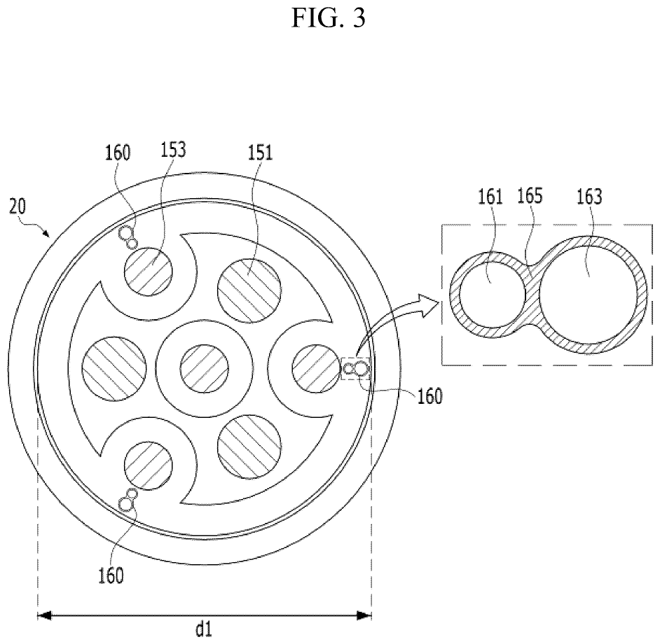

FIG. 3 is a bottom view of the head 20 of the ultrasonic treatment device 100 according to an embodiment of the present disclosure.

Referring to FIG. 2 together, the head 20 may be coupled to one side of the gripping portion 10, and the one surface of the head 20 may be in contact with the user's skin, which is the treatment area. Further, the ultrasonic generator 150 may be located on the one surface of the head 20.

In this regard, the ultrasonic generator 150 may include a first vibrator 151 that generates the high intensity focused ultrasound. With the high intensity focused ultrasound generated by the first vibrator 151, the ultrasonic treatment device 100 according to an embodiment of the present disclosure may be used for a skin lifting or skin tightening procedure, and in particular, may be used for an obesity treatment by burning or melting and decomposing fatty tissues of a subcutaneous fat layer.

Additionally, the ultrasonic generator 150 may include a second vibrator 153 in a planar type. The second vibrator 153 may generate planar ultrasound, and may emulsify fat in the subcutaneous fat layer in the above-described obesity treatment via the planar ultrasound.

That is, a treatment effect of the obesity treatment may be further improved by emulsifying the fat in the subcutaneous fat layer via the second vibrator 153 in the planar type and burning or melting and decomposing the fatty tissues in the subcutaneous fat layer via the first vibrator 151, which generates the high intensity focused ultrasound.

Additionally, the ultrasonic generator 150 may include a plurality of first vibrators 151 and a plurality of second vibrators 153. In this case, the plurality of first vibrators 151 and the plurality of second vibrators 153 may be arranged in various shapes.

The sensor module 160 may include the LED 161, the illuminance sensor 163 disposed adjacent to the LED 161, and a partition wall 165 between the LED 161 and the illuminance sensor 163. In addition, the sensor module 160 may include a plurality of sensor modules located along an outer edge of the one surface of the head 20 and spaced apart from each other.

The sensor module 160 may sense whether the one surface of the head 20 is in contact with the user's skin. In particular, as the plurality of sensor modules 160 are located along the outer edge of the one surface of the head 20 and spaced apart from each other, each of the plurality of sensor modules 160 may sense whether the contact is made, thereby sensing whether the one surface of the head 20 and the user's skin are in complete contact with each other.

That is, the ultrasonic treatment device 100 according to an embodiment of the present disclosure may sense that the one surface of the head 20 and the user's skin are not in complete contact with each other when at least one of the plurality of sensor modules 160 senses a non-contact. In this case, as described above with reference to FIG. 1, the controller 170 may stop the operation of the ultrasonic generator 150.

Additionally, the controller 170 may measure a resistance value of the illuminance sensor 163, which changes depending on a brightness of sensed light, and determine whether the one surface of the head 20 is in contact with the user's skin via a change in the resistance value.

That is, the controller 170 may measure the resistance value of the illuminance sensor 163 of each of the plurality of sensor modules 160, and, when the resistance value of at least one illuminance sensor 163 changes, may determine that the one surface of the head 20 and the user's skin are not in contact with each other and stop the operation of the ultrasonic generator 150.

In addition, the controller 170 may set a sum of the resistance values of the illuminance sensors 163 of the plurality of sensor modules 160, which is for determining that the one surface of the head 20 and the user's skin are in contact with each other, as a reference value, and when the sum of the resistance values measured by the respective illuminance sensors 163 of the plurality of sensor modules 160 has a difference from the reference value, may determine that the one surface of the head 20 and the user's skin are not in contact with each other and stop the operation of the ultrasonic generator 150.

In addition, the one surface of the head 20 may be formed in various shapes. In addition, the one surface of the head 20 may be formed in various sizes considering the treatment area, and in some cases, the one surface of the head 20 may be formed in a limited size.

For example, in the obesity treatment, a user's primary treatment area may be thighs, the abdomen, or buttocks. In this regard, the high intensity focused ultrasound is irradiated by being focused at a depth corresponding to the subcutaneous fat layer in a skin layer of the primary treatment area. Therefore, side effects may occur when the high intensity focused ultrasound is irradiated to a user's face, whose skin layer is relatively thinner than that of the primary treatment area mentioned above.

Therefore, as shown in FIG. 3, a diameter d1 of the one surface of the head 20 may be determined by considering a size and a curvature of the user's face. In other words, by ensuring that the diameter d1 of the one surface of the head 20 is equal to or greater than half the horizontal or vertical length of the user's face, the one side of head 20 may be prevented from being in complete contact with the user's face.

Accordingly, in the ultrasonic treatment device 100 according to an embodiment of the present disclosure, the diameter d1 of the one surface of the head 20 may be about 8 cm or greater considering an average face size of adult women, which is 22.3 cm in the vertical length and 14.7 cm in the horizontal length. Accordingly, when the user brings the ultrasonic treatment device 100 into contact with the face thereof, the one surface may be prevented from being in complete contact with the face, so that the ultrasound may not be irradiated.

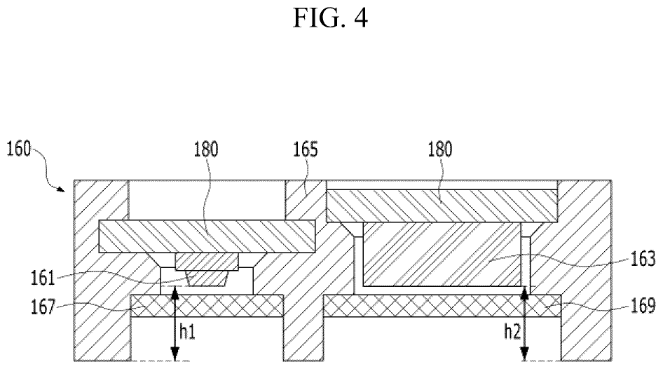

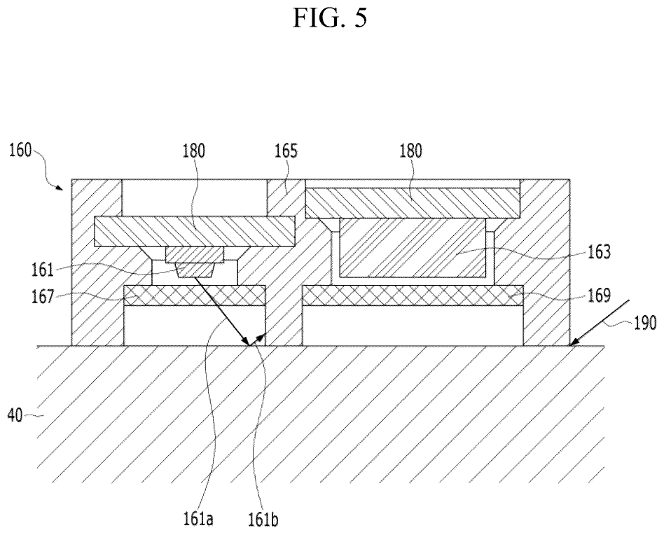

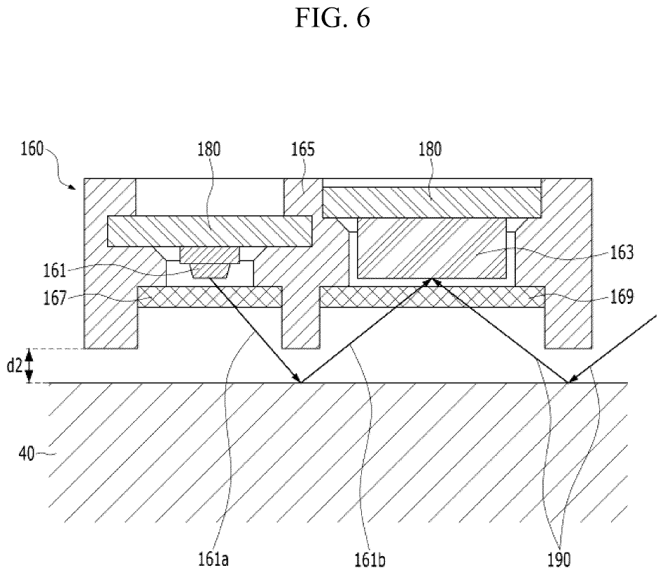

FIG. 4 is a cross-sectional view of the sensor module 160 of the ultrasonic treatment device 100 according to an embodiment of the present disclosure. FIGS. 5 and 6 are diagrams for illustrating a scheme of sensing whether there is a contact with skin 40 via the sensor module 160 of the ultrasonic treatment device 100 according to an embodiment of the present disclosure.

The sensor module 160 may include the LED 161, the illuminance sensor 163 disposed adjacent to the LED 161, and the partition wall 165 between the LED 161 and the illuminance sensor 163. Further, the sensor module 160 may include a printed circuit board 180 on which the LED 161 and the illuminance sensor 163 are electrically connected to each other. That is, the LED 161 and the illuminance sensor 163 may be operated as current is applied thereto via the printed circuit board 180. When the current is applied, the LED 161 emits light, and the illuminance sensor 163 senses light.

Referring to FIG. 3 together, one end of the partition wall 165 in the sensor module 160 may be located on the one surface of the head 20. Further, the LED 161 may be positioned with a vertical level difference h1 from the one end of the partition wall 165. Additionally, the illuminance sensor 163 may be positioned with a vertical level difference h2 from the one end of the partition wall 165.

That is, as shown in FIG. 5, the one end of the partition wall 165 may be located on the one surface of the head 20 and may be in contact with the skin 40. In this case, because the LED 161 and the illuminance sensor 163 are located with the respective vertical level differences h1 and h2 from the one end of the partition wall 165, spaces where the LED 161 and the illuminance sensor 163 are respectively located may be separated from each other by the partition wall 165 and the skin 40.

Accordingly, when the one surface of the head 20 comes into contact with the skin 40, light 161a emitted from the LED 161 is not able to be sensed by the illuminance sensor 163. Likewise, reflected light 161b of the LED 161 is also not able to be sensed by the illuminance sensor 163.

In addition, to separate the above-described spaces where the LED 161 and the illuminance sensor 163 are respectively located from each other and sense whether the skin 40 is in contact, the partition wall 165 may be painted black so as not to affect the sensing, via the illuminance sensor 163, of whether the skin 40 is in contact.

Accordingly, light emitted directly from LED 161 toward the illuminance sensor 163, rather than light emitted from the LED 161 toward the skin 40, may be reliably blocked.

In addition, the partition wall 165 painted in black may reliably block reflected light 161b of the LED 161 or external light 190, which may affect the sensing, via the illuminance sensor 163, of whether the skin 40 is in contact.

In addition, because the ultrasound has a characteristic of being reflected or extinguished when it comes in contact with air, a liquid gel for ultrasound injection is used to increase an effect of an ultrasound treatment. In this case, when the LED 161 and the illuminance sensor 163 are not positioned with the vertical level differences h1 and h2 from the one end of the partition wall 165, the LED 161 and the illuminance sensor 163 may come into contact with the gel for the ultrasonic injection, and in this case, the LED 161 and the illuminance sensor 163 may be damaged.

Therefore, to protect the LED 161 and the illuminance sensor 163 from the gel for the ultrasonic injection, the LED 161 and the illuminance sensor 163 may be positioned with the vertical level differences h1 and h2 from the one end of the partition wall 165, respectively.

In addition, the sensor module 160 may include a first plate-shaped member 167 that is vertically coupled to the partition wall 165 and transmits light 161a emitted from the LED 161 therethrough. In addition, the sensor module 160 may include a second plate-shaped member 169 that is vertically coupled to the partition wall 165 and transmits reflected light 161b of the LED 161 or external light 190 therethrough.

Via the first plate-shaped member 167 and the second plate-shaped member 169, a risk of the LED 161 and the illuminance sensor 163 being damaged by the gel for the ultrasonic injection described above may be prevented. That is, the first plate-shaped member 167 and the second plate-shaped member 169 may perform a waterproof function to protect the LED 161 and the illuminance sensor 163 from the liquid gel for the ultrasonic injection.

In addition, the first plate-shaped member 167 and the second plate-shaped member 169 may include a transparent optical material with a high light transmittance to transmit light 161a emitted from the LED 161, reflected light 161b of the LED 161, or external light 190.

FIG. 6 shows an embodiment in which the sensor module 160 senses the non-contact as there is a separation spacing d2 between the one end of the partition wall 165 and the skin 40.

In other words, because there is the separation spacing d2 between the one end of the partition wall 165 and the skin 40, reflected light 161b of the LED 161 is sensed by the illuminance sensor 163. Additionally, the external light 190 is also sensed by the illuminance sensor 163. Ultimately, because reflected light 161b of the LED 161 and external light 190 are sensed by the illuminance sensor 163, the sensor module 160 may sense the non-contact via the illuminance sensor 163.

Although the skin 40 is shown flat in FIG. 6, the skin 40 may be curved, and in this case, only one of reflected light 161b of the LED 161 and external light 190 may be sensed by the illuminance sensor 163.

Therefore, in the sensor module 160 of the present disclosure, the illuminance sensor 163 may sense the non-contact when at least one of reflected light 161b of the LED 161 and external light 190 is sensed, and thus, may accurately sense whether the skin 40 is in contact.

In addition, the sensor module 160 in the present disclosure does not sense only external light 190 to sense whether the skin 40 is in contact, but also senses reflected light 161b of the LED 161, so that whether the skin 40 is in contact may be accurately sensed even in a dark environment.

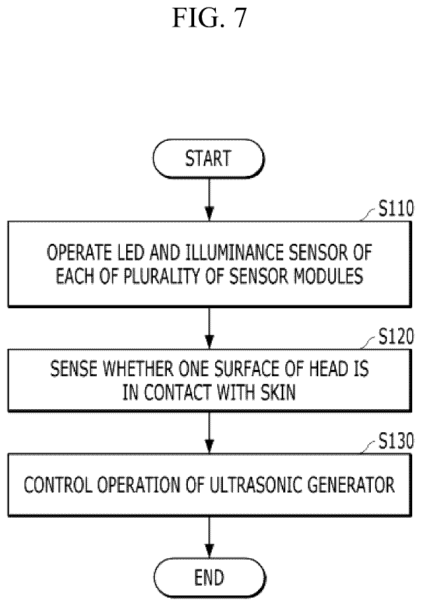

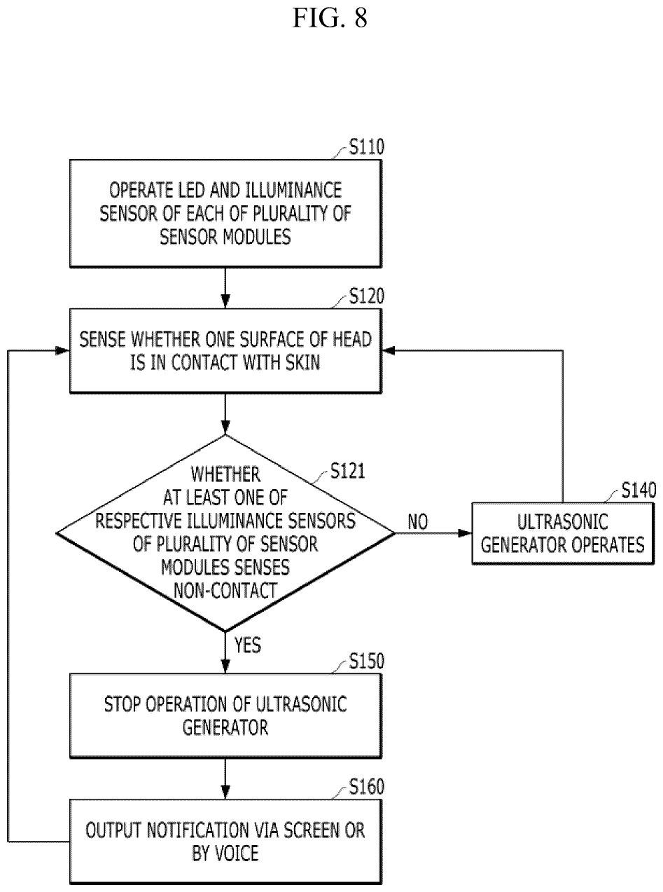

FIG. 7 is a diagram for illustrating a method for controlling the ultrasonic treatment device 100 according to an embodiment of the present disclosure. FIG. 8 is a diagram for illustrating the method for controlling the ultrasonic treatment device 100 in FIG. 7 in more detail.

First, as described above with reference to FIG. 1, the user may turn on the ultrasonic treatment device 100 via the power button 111, set the treatment area, the operation time, the timer setting, the ultrasonic intensity level, and the like via the mode selection button 113, and press the operation button 115. In addition, the LED 161 and the illuminance sensor 163 of each of the plurality of sensor modules 160 may operate (S110).

Whether the one surface of the head 20 is in contact with the user's skin may be sensed via the plurality of sensor modules 160 (S120). Additionally, the operation of the ultrasonic generator 150 may be controlled based on whether the one surface of the head 20 is in contact with the user's skin sensed by each of the plurality of sensor modules 160 (S130). In this regard, the operation of the ultrasonic generator 150 may be controlled based on the operation signal transmitted to the controller 170 based on the settings made by the user described above.

The method for controlling the ultrasonic treatment device 100 according to an embodiment of the present disclosure will be described in more detail with respect to FIG. 8. The LED 161 and the illuminance sensor 163 of each of the plurality of sensor modules 160 may operate (S110), and whether the one surface of the head 20 is in contact with the skin 40 may be sensed via the plurality of sensor modules 160 (S120).

In this regard, the sensor module 160 may include the LED 161, the illuminance sensor 163 disposed adjacent to the LED 161, and the partition wall 165 between the LED 161 and the illuminance sensors 163, as shown in FIGS. 3 to 6. Further, the illuminance sensor 163 may sense the non-contact when at least one of reflected light 161b of the LED 161 and external light 190 is sensed.

In other words, when the one surface of the head 20 and the skin 40 are in contact with each other, the illuminance sensor 163 does not sense reflected light 161b of the LED 161 or external light 190, so that the illuminance sensor 163 may sense the contact. On the other hand, when the one surface of the head 20 and the user's skin 40 are not in contact with each other, the illuminance sensor 163 senses at least one of reflected light 161b of the LED 161 and external light 190, so that the illuminance sensor 163 may sense the non-contact.

Therefore, depending on whether at least one of the respective illuminance sensors 163 of the plurality of sensor modules 160 senses the non-contact (S121), the ultrasonic generator 150 operates only when the non-contact is not sensed (S140).

In addition, in the method for controlling the ultrasonic treatment device 100 according to an embodiment of the present disclosure, to control the operation of the ultrasonic generator 150 by continuously sensing whether the one surface of the head 20 is in contact with the skin 40 during the procedure, after the operation of the ultrasonic generator 150 (S140), whether the one surface of the head 20 is in contact with the skin 40 may be continuously sensed (S120).

When at least one of the respective illuminance sensors 163 of the plurality of sensor modules 160 senses the non-contact, the operation of the ultrasonic generator 150 is stopped (S150). Further, as described above with reference to FIGS. 1 and 2, a notification may be output on a screen or by voice via the display 121 and the audio output unit 123 located in the manipulator 30 (S160). In this regard, the notification may include a notification requesting the user to re-adjust a location of the ultrasonic treatment device 100 because it is sensed that the one surface of the head 20 and the skin 40 are not in contact with each other.

Thereafter, by sensing whether the one surface of the head 20 and the skin 40 are in contact with each other again (S120), the ultrasonic generator 150 may be controlled to operate only when the one surface of the head 20 is in contact with the skin 40 via the above-described steps.

The above detailed description is to be construed in all aspects as illustrative and not restrictive. The scope of the present disclosure should be determined by reasonable interpretation of the appended claims and all changes coming within the equivalency range of the present disclosure are intended to be embraced in the scope of the present disclosure.

What is claimed is:

1. An ultrasonic treatment device comprising:
a gripping portion;
a head coupled to one side of the gripping portion;
an ultrasonic generator located on one surface of the head;
a sensor module configured to sense whether the one surface of the head is in contact with skin; and
a controller configured to control operation of the ultrasonic generator based on whether the one surface of the head is in contact with the skin sensed by the sensor module,
wherein the sensor module includes:
an LED;
an illuminance sensor disposed adjacent to the LED; and
a partition wall located between the LED and the illuminance sensor,
wherein the illuminance sensor senses that the one surface of the head is not in contact with the skin when at least one of reflected light of the LED and external light is sensed.

2. The ultrasonic treatment device of claim 1, wherein the sensor module includes a plurality of sensor modules located along an outer edge of the one surface of the head and spaced apart from each other.

3. The ultrasonic treatment device of claim 2, wherein the controller is configured to stop the operation of the ultrasonic generator when at least one of the respective illuminance sensors of the plurality of sensor modules senses that the one surface of the head is not in contact with the skin.

4. The ultrasonic treatment device of claim 1, wherein the partition wall is painted black so as not to affect the sensing, by the illuminance sensor, of whether the one surface of the head is in contact with the skin.

5. The ultrasonic treatment device of claim 1, wherein the sensor module has the partition wall having one end located on the one surface of the head,
wherein the LED and the illuminance sensor are located with vertical level differences from the one end of the partition wall.

6. The ultrasonic treatment device of claim 5, wherein the sensor module includes:
a first plate-shaped member vertically coupled to the partition wall, wherein the first plate-shaped member transmits light emitted from the LED therethrough; and
a second plate-shaped member vertically coupled to the partition wall, wherein the second plate-shaped member transmits reflected light of the LED or external light therethrough.

7. The ultrasonic treatment device of claim 1, wherein the ultrasonic generator includes a first vibrator configured to generate high intensity focused ultrasound.

8. The ultrasonic treatment device of claim 7, wherein the ultrasonic generator further includes a second vibrator in a planar type.

9. A method for controlling an ultrasonic treatment device, the method comprising:
operating an LED and an illuminance sensor of each of a plurality of sensor modules;
sensing whether one surface of a head is in contact with skin via the plurality of sensor modules; and
controlling operation of an ultrasonic generator based on whether the one surface of the head is in contact with the skin sensed by each of the plurality of sensor modules,
wherein the sensing of whether the one surface of the head is in contact with the skin includes sensing, by the illuminance sensor, that the one surface of the head is not in contact with the skin when at least one of reflected light of the LED and external light is sensed.

10. The method of claim 9, wherein the controlling of the operation of the ultrasonic generator includes stopping the operation of the ultrasonic generator when at least one of the respective illuminance sensors of the plurality of sensor modules senses that the one surface of the head is not in contact with the skin.

11. The method of claim 9, further comprising:
outputting a notification to a user via a screen or by voice when it is sensed that the one surface of the head is not in contact with the skin.
